(19) United States
(12) Patent Application Publication
(10) Pub. No.: US 2025/0316364 A1
(43) Pub. Date: Oct. 9, 2025

(54) COMPUTERIZED METHOD AND SYSTEM FOR ANNOTATING IMAGING SCANS FOR USE IN SURGERY

(71) Applicant: PRECISION HIP, LLC, Winter Haven, FL (US)

(72) Inventors: Juan Alvarez, Winter Haven, FL (US); John Diana, Napa, CA (US); Paul Jeffrey King, Arnold, MD (US)

(73) Assignee: PRECISION HIP, LLC, Winter Haven, FL (US)

(21) Appl. No.: 18/629,669

(22) Filed: Apr. 8, 2024

(51) Int. Cl. G16H 30/40; G06F 3/04845; G06V 10/24; G06V 20/70

(52) U.S. Cl. CPC G16H 30/40; G06F 3/04845; G06V 10/24; G06V 20/70; G06V 2201/033

(57) ABSTRACT

A computerized method and system for annotating imaging scans for use in surgery. The method involves receiving an image that contains a visual representation of an area of a patient's body from a communication device. The image is displayed via a graphical user interface on a computing device that provides a set of graphical tools to a user. The tools allow the user to annotate the image, which can be based at least in part on input to the graphical user interface. An annotation is received relating to one or more portions of the anatomy that appears in the image and is then applied to the image so that it aligns with the relevant anatomy. The method and system can further include artificial intelligence or machine learning modules to generate annotations and identify anatomical features and potential medical defects in the image. The image and annotations can then be used to guide surgical procedures.

### BACKGROUND OF THE INVENTION

In the medical field, especially in the domain of surgery, imaging scans such as X-rays, CT scans, or MRI scans play a critical role. They provide visual representations of the underlying anatomy and structures, aiding physicians in diagnosis, treatment planning, and surgery execution. However, interpreting and understanding these imaging scans requires significant expertise and can be time-consuming. Often, specific key anatomical structures need to be identified and analyzed with precision, which can cause difficulties for even experienced practitioners. Moreover, for complex procedures such as surgeries, these imaging scans need to be annotated with surgical plans or to structure specific indications, consuming considerable time and resources.

To assist in addressing these challenges, embodiments detailed herein propose computerized methods and systems for aiding and assisting users with annotating imaging scans. These systems and methods are particularly useful in surgical settings. In certain embodiments, the inscription and identification process can be powered by machine learning-based algorithms, which can interpret and determine critical anatomical structures, adding a further layer of efficiency and precision to medical imaging analysis. Moreover, the technology can be integrated seamlessly in the existing workflow of the surgeons, assisting them in the accurate interpretation and analysis of the imaging scans. The innovation holds potential in reducing the workload of healthcare professionals, improving the accuracy and efficiency of interpretations, diagnosis, planning, and surgical accuracy, thereby enhancing overall patient care.

### SUMMARY OF THE INVENTION

A computerized method and system for annotating imaging scans for use in surgery. The method involves receiving an image that contains a visual representation of an area of a patient's body from a communication device, such as over a wireless network to the wireless radios in a mobile device. The image is displayed via a graphical user interface on a computing device, such as a mobile device, that provides a set of graphical tools to a user.

In certain embodiments, the tools allow the user to annotate the image, which can be based at least in part on input to the graphical user interface (e.g., capacitive touch screen on a mobile device). An annotation is received relating to one or more portions of the anatomy that appears in the image and is then applied to the image so that it aligns with the relevant anatomy. The method and system can further include artificial intelligence or machine learning modules to generate annotations and identify anatomical features and potential medical defects in the image. The image and annotations can then be used to guide surgical procedures.

According to an embodiment of the present invention, a computerized system and method for annotating imaging scans for use in surgery comprises the steps of: receiving, from a communication device, an image, wherein the image contains a visual representation of an area of a body of a patient; displaying, via a graphical user interface on a computing device, the image; providing to a user, via the graphical user interface on the computing device, a set of graphical tools, wherein the set of graphical tools allow the user to annotate the image; receiving an annotation, based at least in part on input to the graphical user interface, wherein the annotation relates to one or more portions of anatomy appearing in the image; and applying the annotation to the image in a manner such that the annotation is aligned with a portion of anatomy appearing in the image.

In certain embodiments, the image is an x-ray image. In other embodiments, the image could be any other form of imaging results from a scan, such as an MRI or CT scan.

Embodiments of the present invention can be used on any region or area of a patient's body. In one preferred embodiment, embodiments of the present invention may be used to identify one or more hips of the patient, and assist in surgical alignment and repair of the hips. Areas of the hips that could be identified by embodiments of the present invention include, but are not limited to, an ilium, an ischium, a pubis, a femoral head, an acetabulum, a femoral neck, a greater trochanter, a lesser trochanter, and a pelvic line.

According to an embodiment of the present invention, the computerized systems and methods may further comprise the step of: generating, via an artificial intelligence or machine learning powered module enabled with image analysis capabilities, one or more annotations for the one or more portions of the anatomy.

According to an embodiment of the present invention, the computerized systems and methods may further comprise the step of: identifying, via the artificial intelligence or machine learning powered module enabled with image analysis capabilities, the one or more portions of the anatomy, and potential medical defects appearing in the image related to the one or more portions of the anatomy.

According to an embodiment of the present invention, the artificial intelligence or machine learning powered module may be, but is not limited to: machine learning models trained on various amounts of test and training data, neural networks, artificial neural networks (ANN), convolution neural networks (CNN), recurrent neural networks (RNN), deep learning models and deep-learning-based generative models, and generative adversarial networks (GANs).

According to an embodiment of the present invention, the computerized systems and methods may further comprise the step of: using the image as augmented by the annotation to properly align the one or more portions of the anatomy of the patient.

According to an embodiment of the present invention, the computerized systems and methods may further comprise the steps of: identifying, via an image analysis module, a plurality of anatomical components in the image; and placing annotations on the image associated with each of the plurality of anatomical components.

### BRIEF DESCRIPTION OF DRAWINGS

Turning now descriptively to the drawings, in which similar reference characters denote similar elements throughout the several views, the figures illustrate a few exemplary embodiments of the present invention. With regard to the reference numerals used, the following numbering is used throughout the various drawing figures.

FIG. 1 is a schematic illustration of an exemplary computing device, in accordance with at least some exemplary embodiments of the present invention;

FIG. 2 is a schematic illustration of an exemplary network, in accordance with at least some exemplary embodiments of the present invention;

FIG. 3 is an illustration of an exemplary method for annotating image scans for use in surgery;

FIG. 4 shows an exemplary method for annotating image scans for use in surgery;

FIG. 5 is an illustration of an exemplary method for annotating image scans for use in surgery;

FIG. 6 is an illustration of an exemplary method for annotating image scans for use in surgery; and

FIG. 7 is an illustration of a graphical user interface showing annotated portions of anatomy of a hip, in accordance with an embodiment of the present invention.

### DETAILED DESCRIPTION OF THE INVENTION

The following discussion describes in detail one embodiment of the invention (and several variations of that embodiment). This discussion should not be construed, however, as limiting the invention to those particular embodiments, practitioners skilled in the art will recognize numerous other embodiments as well. For definition of the complete scope of the invention, the reader is directed to appended claims.

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

This invention now will be described more fully hereinafter with reference to the accompanying drawings, in which exemplary embodiments are shown. Various embodiments are now described with reference to the drawings, wherein such as reference numerals are used to refer to such as elements throughout. In the following description, for purposes of explanation, numerous specific details are set forth in order to provide a thorough understanding of one or more embodiments. It may be evident, however, that such embodiment(s) may be practiced without these specific details. In other instances, well-known structures and devices are shown in block diagram form in order to facilitate describing one or more embodiments.

This invention may, however, be embodied in many different forms and should not be construed as limited to the embodiments set forth herein. These embodiments are provided so that this disclosure will be thorough and complete and will fully convey the scope of the invention to those of ordinary skill in the art. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Thus, for example, it will be appreciated by those of ordinary skill in the art that the diagrams, schematics, illustrations, and the such as represent conceptual views or processes illustrating systems and methods embodying this invention. The functions of the various elements shown in the figures may be provided through the use of dedicated hardware as well as hardware capable of executing associated software. Similarly, any switches shown in the figures are conceptual only. Their function may be carried out through the operation of program logic, through dedicated logic, through the interaction of program control and dedicated logic or other means, the particular technique being selectable by the entity implementing this invention. Those of ordinary skill in the art further understand that the exemplary hardware, software, processes, methods, and/or operating systems described herein are for illustrative purposes and, thus, are not intended to be limited to any particular named manufacturer.

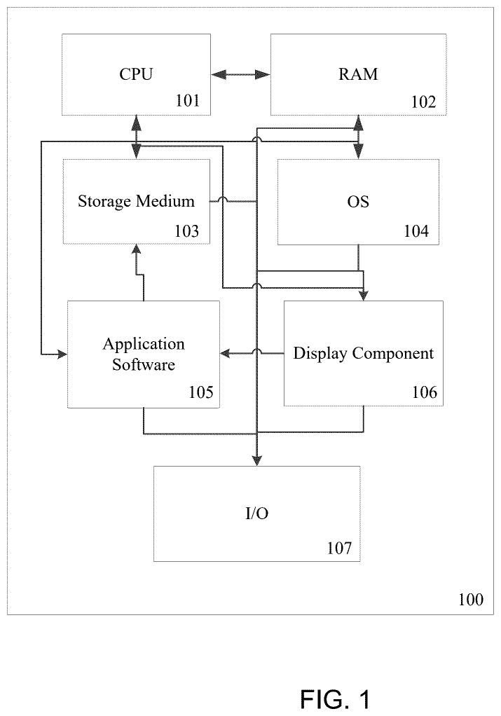

Turning now to FIG. 1, an illustrative representation of a computing device appropriate for use with embodiments of the system of the present disclosure is shown. The computing device 100 can generally be comprised of a Central Processing Unit (CPU, 101), optional further processing units including a graphics processing unit (GPU), a Random Access Memory (RAM, 102), a mother board 103, or alternatively/additionally a storage medium (e.g., hard disk drive, solid state drive, flash memory, cloud storage), an operating system (OS, 104), one or more application software 105, a display element (e.g., monitor, capacitive touchscreen) 106, and one or more input/output devices/means 107, including one or more communication interfaces (e.g., RS232, Ethernet, Wifi, Bluetooth, USB). Useful examples include, but are not limited to, personal computers, servers, tablet PCs, smartphones, or other computing devices. In preferred embodiments of the present invention, multiple computing devices can be operably linked to form a computer network in a manner as to distribute and share one or more resources, such as clustered computing devices and server banks/farms.

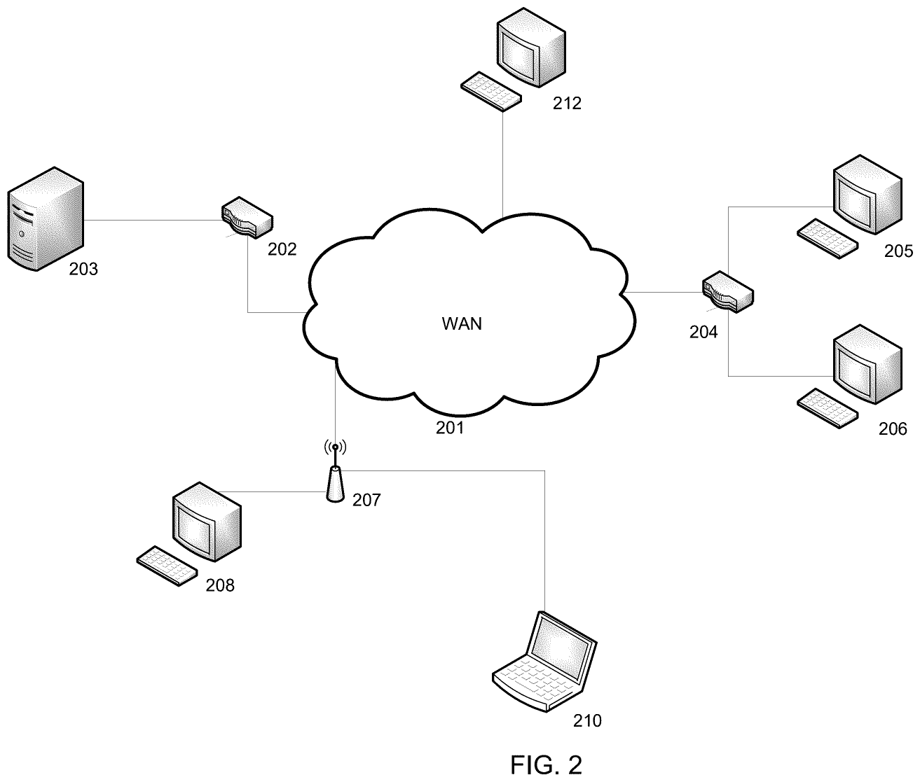

Various examples of such single-unit and multi-unit computer networks suitable for embodiments of the disclosure, their typical configuration and many standardized communication links are well known to one skilled in the art, as explained in more detail and illustrated by FIG. 2, which is discussed herein-below.

According to an exemplary embodiment of the present disclosure, data may be transferred to the system, stored by the system and/or transferred by the system to users of the system across local area networks (LANs) or wide area networks (WANs). In accordance with the previous embodiment, the system may be comprised of numerous servers, mining hardware, computing devices, or any combination thereof, communicatively connected across one or more LANs and/or WANs. One of ordinary skill in the art would appreciate that there are numerous manners in which the system could be configured and embodiments of the present disclosure are contemplated for use with any configuration.

Referring to FIG. 2, a schematic overview of a system in accordance with an embodiment of the present disclosure is shown. The system is comprised of one or more application servers 203 for electronically storing information used by the system. Applications in the server 203 may retrieve and manipulate information in storage devices and exchange information through a WAN 201 (e.g., the Internet). Applications in server 203 may also be used to manipulate information stored remotely and process and analyze data stored remotely across a WAN 201 (e.g., the Internet).

According to an exemplary embodiment, as shown in FIG. 2, exchange of information through the WAN 201 or other network may occur through one or more high speed connections. In some cases, high speed connections may be over-the-air (OTA), passed through networked systems, directly connected to one or more WANs 201 or directed through one or more routers 202. Router(s) 202 are completely optional and other embodiments in accordance with the present disclosure may or may not utilize one or more routers 202. One of ordinary skill in the art would appreciate that there are numerous ways server 203 may connect to WAN 201 for the exchange of information, and embodiments of the present disclosure are contemplated for use with any method for connecting to networks for the purpose of exchanging information. Further, while this application refers to high speed connections, embodiments of the present disclosure may be utilized with connections of any speed.

Components or modules of the system may connect to server 203 via WAN 201 or other network in numerous ways. For instance, a component or module may connect to the system i) through a computing device 212 directly connected to the WAN 201, ii) through a computing device 205, 206 connected to the WAN 201 through a routing device 204, or iii) through a computing device 208, 210 connected to a wireless access point 207. One of ordinary skill in the art will appreciate that there are numerous ways that a component or module may connect to server 203 via WAN 201 or other network, and embodiments of the present disclosure are contemplated for use with any method for connecting to server 203 via WAN 201 or other network. Furthermore, server 203 could be comprised of a personal computing device, such as a smartphone, acting as a host for other computing devices to connect to.

The communications means of the system may be any circuitry or other means for communicating data over one or more networks or to one or more peripheral devices attached to the system, or to a system module or component. Appropriate communications means may include, but are not limited to, wireless connections, wired connections, cellular connections, data port connections, Bluetooth® connections, near field communications (NFC) connections, or any combination thereof. One of ordinary skill in the art will appreciate that there are numerous communications means that may be utilized with embodiments of the present disclosure, and embodiments of the present disclosure are contemplated for use with any communications means.

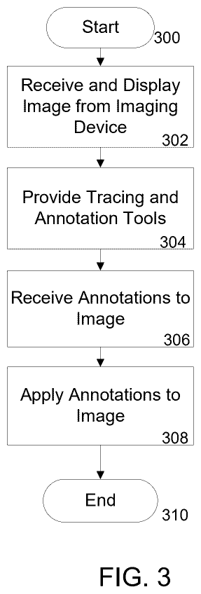

FIG. 3 is an illustration of an exemplary method for annotating image scans for use in surgery. The process starts at step 300 with a user engaging a computing device, such as a smartphone, for the purpose of annotating an image scan. At step 302, the computing device receives an image from an imaging device. In certain embodiments, the image may be received directly from the imaging device. In other embodiments, the imaging device, such as an MRI, CT Scanner or X-Ray Machine, provides the image to a computing device associated with the imaging device, and that computing device handles sending the image to the computing device of the user. The transmission of the image may also involve intermediary networks and computing devices, such as wireless or wired networks, cloud computing devices, servers, or any other well-known method for transferring images. One of ordinary skill in the art would appreciate that there are numerous methods for transferring the image to the computing device of the user, and embodiments of the present invention are contemplated for use with any such methods.

At step 304, the user's computing device displays the image on a graphical user interface (GUI) and is provided a set of tracing and annotation tools. Tracing and annotation tools can include, but are not limited to, shapes, text, freeform drawing tools, image editing tools (e.g., brightness editor, sharpness editor, cropping tool, rotation tool), or any combination thereof. In certain embodiments, the shapes tools may include, but are not limited to, shapes that approximate common anatomical features, or contours associated with common anatomical features, such as the shape or a contour of a side/edge/portion of an ilium, an ischium, a pubis, a femoral head, an acetabulum, a femoral neck, a greater trochanter, or a lesser trochanter.

At step 306, the system receives annotations to the image. In certain embodiments, the annotations are produced by a user interacting with an input device (e.g., capacitive touchscreen, mouse, digital input pad) on the computing device. At step 308, the system processes the annotation data and applies the annotations to the image. At this point, the process ends with the end result being the production of a finalized annotated image for use by the user, for instance in aiding in a surgical procedure.

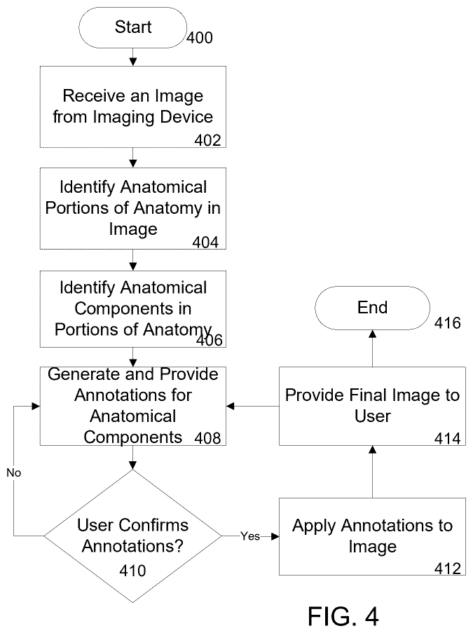

FIG. 4 shows an exemplary method for annotating image scans for use in surgery. The process starts at step 400 with the system being engaged to start the image annotation process. At step 402, the system receives an image from an imaging device, such as an X-Ray machine, CT Scanner or MRI. At step 404, the system identifies anatomical portions of anatomy in the image, such as identifying the area in the image is are the hips or hip of a patient. In certain embodiments of the present invention, the system may be configured to automatically identify the anatomical portions, such as via a machine learning module trained on data sets related to images of various anatomical portions of anatomy.

At step 406, the system identifies the components of the anatomy that are visible in the anatomical portion shown in the image. In certain embodiments of the present invention, the system may be configured to automatically identify the components, such as via a machine learning module trained on data sets related to images of various anatomical components. For instance, the system may automatically identify one or more of an illum, a greater trochanter, a lesser trochanter, an ischium and a pelvic line.

Once identified, the system is configured to generate and provide annotations related to the components of anatomy and provide the initial annotations to the user. At this point (step 410) the user can confirm or alter the annotations. If they are not acceptable, the user can send its alterations back to the system to be generated correctly (step 408). If the annotations are acceptable, the process moves to step 412, where the system applies the annotations to the images. At step 414, the final annotated image is presented to the user and the process ends at step 416.

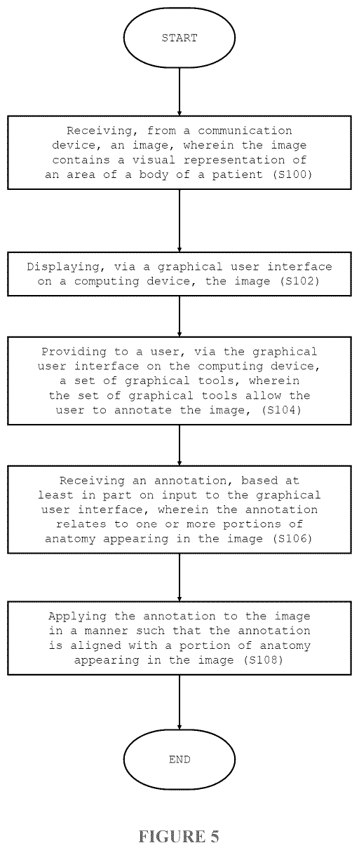

FIG. 5 shows an exemplary illustration of an exemplary method for annotating imaging scans, in accordance with an embodiment of the invention. The method begins by receiving, from a communication device, an image, wherein the image contains a visual representation of an area of a body of a patient S101; generating a two dimensional (2-d) or three-dimensional (3-d) model based on the image, wherein the model contains visual representations of anatomical components of the area of the body S102; allowing a user to manipulate the model, wherein the manipulation includes rotating the model and zooming in and out of the model S103; storing the model in a memory device S104; and displaying the model to the user S105.

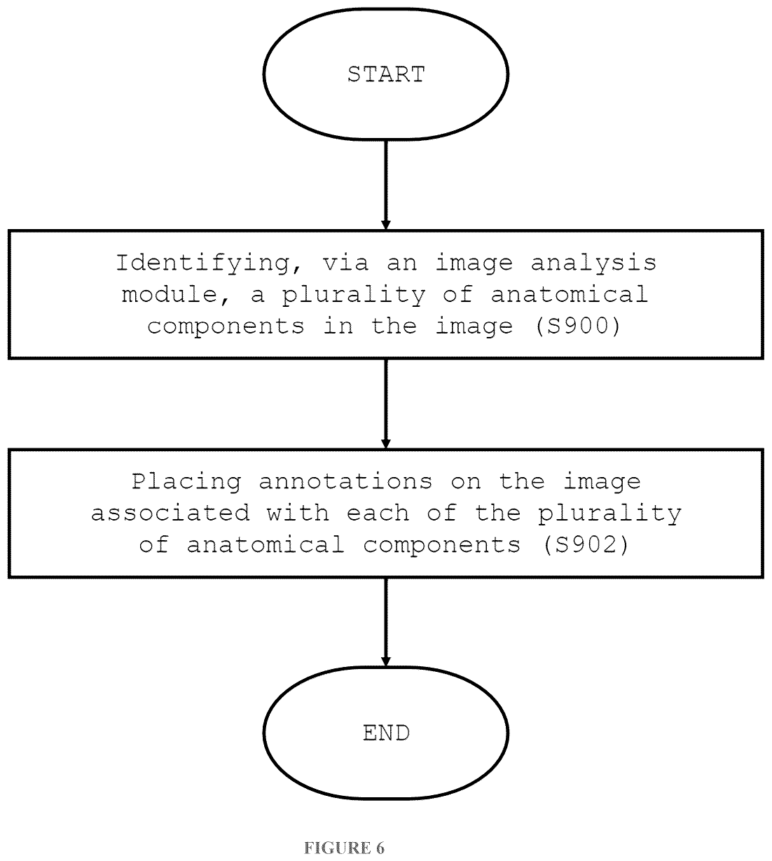

FIG. 6 shows an exemplary illustration of an exemplary method for annotating imaging scans, in accordance with an embodiment of the invention, and builds on FIG. 5. The method starts with the system automatically identifying, via an image analysis module, a plurality of anatomical components in the image (s900). At s902, the system automatically places annotations on the image associated with the identified plurality of anatomical components. At this point, the image is displayed or provided to the user, and the process terminates.

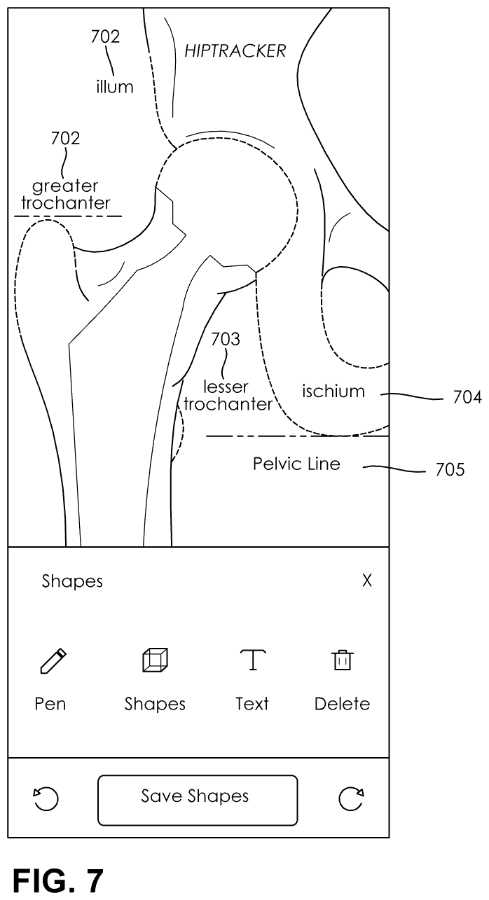

FIG. 7 is an exemplary illustration of a graphical user interface with could be used with embodiments of the present invention. Here, an image of a patient's hip is shown with annotations highlighting the illum 701, the greater trochanter 702, the lesser trochanter 703, the ischium 704 and the pelvic line 705. FIG. 7 also shows the interface where a user can select various tools, such as shapes, text, pencil and other tools to input annotations on the image.

Displaying, via a graphical user interface on a computing device, the image of the anatomical region, the system also allows for virtual dissection and interactive manipulation of the anatomical structure. The user can select, move, rotate, zoom in, or zoom out various components of the model. This offers a far more comprehensive understanding of the internal architecture of the structure, enabling an improved assessment of the patient's condition. Additionally, users can also integrate additional medical information such as CT or MRI scans into the model to further their comprehension. In summary, this computerized system provides a powerful, interactive tool for detailed modeling and visualization of complex skeletal structures, allowing users to manipulate the model and integrate various types of medical data for enhanced diagnosis and treatment. By offering dynamic customizability and an array of perspectives, this system is expected to significantly improve the medical profession's ability to effectively address and manage anatomical problems.

According to an embodiment of the present invention, the system may be configured to provide to a user, via the graphical user interface on the computing device, a set of graphical tools, wherein the set of graphical tools allow the user to annotate the image, and draw objects, lines, shapes and other indicators within the image. The system may also provide the user with the ability to manipulate the image in a 2-dimensional or 3-dimensional plane. In still further embodiments, the system may be configured to allow the user to add points of interest within the image, and adjust lighting and color within the image.

According to an embodiment of the present invention, the system may be configured to receive an annotation, based at least in part on input to the graphical user interface. The annotation generally relates to one or more portions of patient's anatomy appearing in the image. In addition to its core functionality, this ability to annotate images in this fashion provides additional features to increase user efficiency. The graphical user interface is highly intuitive and includes interactive elements, such as drag-and-drop functionality for faster manipulation of anatomical components appearing in the image. Annotation features allow users to tag individual components and mark regions of interest, enabling the rapid and precise identification of areas of interest. For instance, when used in conjunction with hip repair, replacement or alignment surgeries, the annotations may help surgeons correctly align implants, such as a hip replacement, by allowing the surgeon to identify proper alignment of various anatomical features, such as an ilium, an ischium, a pubis, a femoral head, an acetabulum, a femoral neck, a greater trochanter, a lesser trochanter, and a pelvic line.

In addition, an AI-driven identification and annotation tool allows for automated identification and annotation of certain anatomical structures, further expediting the process. This comprehensive suite of features allows for more efficient navigation of the model and expedites the entire analysis process. The AI-driven identification and annotation tool may apply annotations to the image in a manner such that the annotations are aligned with portions of anatomy appearing in the image. In preferred embodiments, the system's core functions involve the collection, integration, and manipulation of digital images, which are analyzed and converted into an interactive virtual model (2-d and/or 3-d). This model may be further annotated to accurately represent anatomical structures.

Once the model is generated, users can rotate and move it from various angles to better understand the anatomical region in question and confirm proper alignment. Additionally, the system enables individual customization, allowing users to add, alter, or remove components of the model as needed. The comprehensive view of the anatomy provided by this system, combined with the individual customization option, makes it highly useful in diagnosing and providing graphical resources for various surgical procedures. The high-quality annotated digital images generated by the system, sophisticated rendering techniques, data analysis capabilities, and dynamic model customization offer a marked improvement over traditional methods such as manual analysis of static images provided by various imaging devices.

FIG. 6 shows an exemplary illustration of method in accordance with an embodiment of the present invention. The method starts with identifying, via an image analysis module, a plurality of anatomical components in the image. The image analysis module can employ computer vision and machine learning techniques to identify the various anatomical components that appear in the image. This module can be configured to use algorithms that can detect and classify features in the image, such as bones, organs, vessels, and other relevant structures. The identified features are then mapped to a standardized representation of the anatomy, allowing the system to automatically annotate the image with labels for each anatomical component. Generating, via the annotation module, an annotation for at least one of the plurality of anatomical components in the image. The annotation module is configured to generate annotations for the various anatomical components identified by the image analysis module. The annotations can include information about the type of feature, its size and shape, any abnormalities present, or other relevant data. The annotations can also include graphical markers that highlight the relevant features, as well as arrows and lines that provide directionality to indicate how the anatomical components relate to each other and how they are aligned or should actual be aligned in a normal operational anatomical component.

The system is then configured to associate the annotation with the anatomical component in the image. In certain embodiments, an image mapping module is responsible for aligning the annotation with the relevant anatomical component in the image. The module can use computer vision system image analysis methodologies and techniques to ensure that the annotation is placed in the correct location, accounting for any differences in orientation or scale between the annotation and the anatomical component. This allows the annotations to be accurately displayed in relation to the underlying anatomy.

Placing annotations on the image associated with each of the plurality of anatomical components may include any number of approaches. For example, the user may select one or more points on the image corresponding to an anatomical component, or may use a combination of graphical tools such as lines, arrows, circles, and/or other shapes to delineate the anatomical component. In some embodiments, the graphical tools may include a pre-defined library of shapes that can be applied to the image. In certain embodiments, the annotations placed on the image may also include annotations relating to medical conditions or defects associated with one or more of the anatomical components. These annotations may be based on user input or generated automatically by a machine learning-based module, for example. The annotations may be in the form of text, numerical data, graphical symbols, or any other type of annotation. Once the annotations have been applied to the image, the annotations may be used to guide a surgical procedure, for example.

In certain embodiments, the annotations may be used to inform the surgeon about potential risks associated with a given surgical approach, or may provide information about a particular anatomical component that may be of interest during the procedure. The annotations may also be used to provide real-time feedback to the surgeon, for example to help the surgeon accurately position instruments or make decisions during the procedure.

According to one embodiment of the present invention, a computerized method for annotating imaging scans used for surgical procedures is utilized. This is facilitated by receiving images portraying a portion of the body of a patient from a communication device. The images that are obtained provide the first level of information about the area of concern that will be under surgical intervention. The images serve as the primary basis for the physicians to make the necessary decisions regarding the medical operation. Such images are vital tools in understanding the underlying complications effectively.

According to an embodiment of the present invention, the graphical tools act as a support system to the user, assisting them in annotating the images to their needs. It facilitates a deeper understanding of the images, offers a chance to mark the vital areas of focus and any potential complications. The graphical tools simplify the process of investigating the images, paving the way for a more precise execution of the surgery.

This invention is sophisticated and can contribute immensely to the medical field, especially for surgeons. The ability to receive an image, annotate it with the help of graphical tools, and apply the annotations to the image can be highly beneficial for doctors and surgeons. This could significantly enhance the precision and accuracy of surgical procedures, reducing potential risks and ensuring a safer and more successful operation, as well as reducing the time it takes to produce high quality annotated images for use in conjunction with the surgeries.

In conclusion, the implementation of this invention in hospitals, clinics, and other medical institutions could revolutionize the way surgeries are planned and carried out. By digitizing, simplifying, and enhancing the process of image-annotation, the invention has the potential to make surgical procedures more efficient, reliable, and result-oriented.

The invention claimed pertains to a computerized method that deals specifically with various medical imaging scans, such as x-ray images. The process deals with enhancing the efficiency and accuracy of interpretation of these images. This optimization is achieved by exploiting innovative algorithms and machine learning protocols that serve to revolutionize the way medical professionals analyze and understand x-ray images. The automated technology utilized in certain embodiments of this invention can play a crucial role in various sectors of the healthcare industry, contributing specifically to radiography, oncology, orthopedics, and other areas that largely depend on medical imaging scans. The objective lies not only in improving detection and diagnostic performance but also in reducing the manual workload, thus potentially speeding up the patient's treatment process.

According to an embodiment of the present invention utilizing computerized analysis systems, a first aspect focuses on the employment of advanced computational techniques and algorithms to analyze medical imaging scans. These algorithms are programmed to identify, characterize, and quantify patterns and features from the images that may not be perceptible to the human eye or may be time consuming to identify manually. This element of the invention ensures a more accurate, comprehensive, and efficient analysis of the images. Moreover, the use of machine learning in this implementation allows the system to improve its performance over time by learning from past imaging data and diagnostics. This guarantees that the effectiveness of the technology only enhances as more data are accumulated and incorporated into the system.

According to an embodiment of the present invention utilizing computerized analysis systems, a second part involves a user-friendly, intuitive interface that allows healthcare personnel to interact with the system effectively. The interactive platform provides the users with the capacity to input patient data, adjust parameters or settings of algorithms if needed, view analyzed images, and interpret the generated data. By integrating this invention into practical caregiving, medical professionals can focus more on the actual treatment of diseases or conditions by relying on the accurate and efficient diagnostic outcomes provided by the system.

In one embodiment of the present invention, a computerized method specifically for analyzing the area of the body of a patient, precisely focusing on one or more hips of the patient. This innovative procedure deftly integrates advanced computer-based techniques, thereby transforming the standard medical approach of patient body area examination, particularly hip evaluation. The invention's unique framework is designed to meticulously evaluate, diagnose, monitor, and manage the health-related issues of the patient, predominantly associated with hips.

Certain embodiments of the present invention involves the use of cutting-edge, computerized radiologic technologies such as Magnetic Resonance Imaging (MRI), Computerized Tomography (CT) scans, and advanced applications for processing the gathered data for evaluating the hip or hips of the patient. Unlike traditional methods, this computerized method enhances precision, reduces human error, and facilitates quicker and accurate decision-making. The method is comprehensive, encapsulating various mechanisms for early disease detection, progression monitoring, treatment efficiency evaluation, and prognosis following therapeutic interventions for conditions, particularly related to the hip area. The method incorporates adaptive algorithms, which dynamically adjust to the patient's subjective variables and creates an objective medical profile, substantially contributing towards personalized medicines and surgeries.

Moreover, this invention adds an unprecedented edge to the ergonomic aspect of patient care. It paves the way for remote medical consultation and examination, thereby overcoming the physical barriers, particularly crucial in scenarios like rehabilitation following hip surgeries wherein constant monitoring becomes paramount. From a technical perspective, certain embodiments of the invention entails the implementation of Machine Learning (ML) and Artificial Intelligence (AI) for advanced image analysis and interpretation, thereby contributing to robust, comprehensive, and efficient patient care. It must be noted that certain embodiments of the present invention are focused on the hips of the patient elevates the current clinical standards, revolutionizes medical experiences, and holds substantial potential to shape the future trajectory of hip health management.

The invention comprises a computerized method for the selection and identification of one or more portions of the anatomy. This is particularly pertinent to the musculoskeletal system, focusing on regions that forms part of the hip joint, pelvic region and related structures in the human body. The regions of anatomy that can be selected include, but are not limited to, an ilium, which is the broad, flared portion of the pelvis, an ischium, the curved bone forming the base of each half of the pelvis, and a pubis, which is the medial anterior portion of the pelvis. In certain embodiments, these structure determinations are facilitated through an interactive database and visualization system that enables the user to select, isolate and interactively work with these anatomical structures in a virtual environment.

In other embodiments, structure determinations are identified via an AI/ML model, such as a machine learning model trained on data related to these structure determinations. According to certain embodiments of the present invention, precision of these models is reinforced via its consideration of the hip joint specific structures. This includes a femoral head, referring to the rounded top part of the femur that fits into the acetabulum to form the hip joint. The femoral neck, the region just below the femoral head that connects the head to the shaft of the femur, is also selected and identified by this computerized method. In addition, the greater and lesser trochanter, referring to the two bony prominences at the proximal end of the femur, can also be selected using this system. The acetabulum, which is the socket in the pelvis that the femoral head fits into, is also selectable. This ensures a comprehensive and detailed analysis and visualization of the structures that are most critical to hip joint health and function.

Additionally, certain embodiments of the computerized method allow for the selection and identification of the pelvic line. This key anatomical landmark, which represents a line drawn through the symphysis pubis and the sacroiliac joint, providing a baseline for assessing pelvic symmetry and alignment. The implementation of this technologically-driven visualization and identification system increases precision in the medical field, specifically in orthopedic diagnostics and treatment planning, by ensuring access to accurate, illustrative, and interactive details of these critical skeletal structures. This versatile tool seamlessly merges the specifics of anatomy with the functionality of computerized systems to redefine and enhance the practice of medical sciences.

Certain embodiments of the present invention rely on elaborate and effective AI or ML algorithms that are capable of accurately identifying and annotating various aspects of the anatomy from medical imaging data. The module may be configured to process the images, recognize various anatomical structures and generates relevant annotations. These annotations may include, but not limited to, labels, outlines, measurements, classifications, and other data pertinent to the structure under consideration.

In addition to enhancing interpretation, these annotations also significantly facilitate in communicating analytical information in a precise, meaningful, and easily understandable format. The AI module's ability to process a multitude of images swiftly and accurately, presents an effective tool for radiologists, physicians, and other medical practitioners in their examination, diagnosis, and treatment planning processes. Furthermore, it significantly reduces manual image processing time, improves accuracy, and augments efficiency in the clinical workflow. This invention thus synergizes AI, machine learning technologies with clinical practices to advance medical image analysis and improve overall healthcare service quality.

The present invention provides a computerized method for identifying regions of interest in anatomic structures via the use of artificial intelligence or machine learning. The invention is directed towards an intelligent module with image analysis capabilities designed to detect, classify, and interpret medical image data in order to assist clinical decision-making. The system is engineered to process medical images in a variety of formats, ranging from radiographic or magnetic resonance imaging (MRI) scans to microscopic images. The system utilizes an advanced algorithm, built upon artificial intelligence (AI) or machine learning (ML) principles, which enables it to identify specific anatomical structures, and further predict potential medical defects or anomalies appearing within these structures.

In addition, the technology embedded in this invention allows for continuous learning and adaptation. As the system interacts with more data, the machine learning algorithm refines its analytical processes, becoming increasingly astute at identifying anatomical portions and detecting potential medical defects. This ensures the system remains up-to-date with advancements in medicine and pathology, complete with the ability to handle complex and unfamiliar medical scenarios. Overall, the invention contributes to the field of healthcare by equipping it with a high-performing, intelligent tool for medical image analysis and interpretation, thus assisting physicians in making informed clinical decisions.

In certain embodiments, a specialized module, which is powered by artificial intelligence (AI) or machine learning is utilized. The module is selected from a diverse group of machine learning models, neural networks, and deep learning models. This includes machine learning models that have undergone training on varying amounts of test and training data. The key characteristic of these models is their ability to learn and improve from experience, adapting to new inputs while allowing the system to automatically improve its performance as more data is processed.

The group also comprises of different neural network models such as artificial neural networks (ANN), convolution neural networks (CNN), and recurrent neural networks (RNN). ANNs simulate the behavior of human neurons in processing data, while CNNs are predominantly used in image and video processing and use a variant of multilayer perceptrons. On the other hand, RNNs are used for analyzing sequential data and learning patterns, making them useful for processes such as handwriting and speech recognition. They are designed to recognize patterns across time, thus useful in time-series prediction applications.

The system may also be configured to utilize deep learning models and deep-learning-based generative models. These models operate by mimicking the function and structure of brain neural networks and are proficient in process high-dimensional data, such as images and videos. Among the options available, the system particularly acknowledges the inclusion of generative adversarial networks (GANs). GANs consist of two models where one generates candidates while the other evaluates them, thus allowing the system to create believable artificial outputs. By encompassing the advanced capabilities of these AI powered components, the invention enables robust analysis and processing of complex data for a variety of uses.

In certain embodiments, the system features the capability to overlay these annotated images onto the actual patient's anatomy in real-time, thereby providing a clear and readable visual aid to medical practitioners in aligning the various parts of the patient's anatomy in the correct orientation. Importantly, the innovative feature of the system is the advanced processing it uses to display the augmented image, translated into three dimensions, and placing the image in the same spatial i.e., stereoscopic perspective as the actual anatomy. Furthermore, the augmented reality image, with the annotations, can be manipulated by the user by rotating, zooming, or tilting in order to get low, high, or side views of the patient's anatomy, thereby enhancing the interaction and ability to properly align the anatomy.

In addition to this, the system leverages state-of-the-art machine learning and artificial intelligence methods to develop an intelligent annotation system, intelligently determining the best way to guide the user to align the anatomy using these annotated images. The system is capable of doing this by learning from previous alignment actions and adjusting the annotation guidance accordingly. The system is designed to intuitively incorporate feedback loops for iterative improvement.

Furthermore, the system also applies the annotations to the image in a manner where the notes are aligned clearly with the portion of the anatomy being referenced. This ensures that any marked or annotated features are readily identifiable on the image, allowing the user to be accurately guided by these annotations during the surgery.

Various modifications and alterations of the invention will become apparent to those skilled in the art without departing from the spirit and scope of the invention, which is defined by the accompanying claims. It should be noted that steps recited in any method claims below do not necessarily need to be performed in the order that they are recited. Those of ordinary skill in the art will recognize variations in performing the steps from the order in which they are recited. In addition, the lack of mention or discussion of a feature, step, or component provides the basis for claims where the absent feature or component is excluded by way of a proviso or similar claim language.

While various embodiments of the present invention have been described above, it should be understood that they have been presented by way of example only, and not of limitation. The various diagrams may depict an example architectural or other configuration for the invention, which is done to aid in understanding the features and functionality that may be included in the invention. The invention is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the present invention. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the invention, whether or not such embodiments are described and whether or not such features are presented as being a part of a described embodiment. Thus the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the such as; the term “example” is used to provide exemplary instances of the item in discussion, not an exhaustive or limiting list thereof; the terms “a” or “an” should be read as meaning “at least one,” “one or more” or the such as; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Hence, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated.

The presence of broadening words and phrases such as “one or more,” “at least,” “but not limited to” or other such as phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, may be combined in a single package or separately maintained and may further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

1. A computerized method for annotating imaging scans for use in surgery, the method comprising:
receiving, from a communication device, an image, wherein the image contains a visual representation of an area of a body of a patient;
displaying, via a graphical user interface on a computing device, the image;
providing to a user, via the graphical user interface on the computing device, a set of graphical tools, wherein the set of graphical tools allow the user to annotate the image;
receiving an annotation, based at least in part on input to the graphical user interface, wherein the annotation relates to one or more portions of anatomy appearing in the image;
applying the annotation to the image in a manner such that the annotation is aligned with a portion of anatomy appearing in the image.

2. The computerized method of claim 1, wherein, the image is an x-ray image.

3. The computerized method of claim 1, wherein the area of the body of a patient is one or more hips of the patient.

4. The computerized method of claim 3, wherein the one or more portions of the anatomy is selected from the group, comprising:
an ilium, an ischium, a pubis, a femoral head, an acetabulum, a femoral neck, a greater trochanter, a lesser trochanter, and a pelvic line.

5. The computerized method of claim 4, further comprising the step of generating, via an artificial intelligence or machine learning powered module enabled with image analysis capabilities, one or more annotations for the one or more portions of the anatomy.

6. The computerized method of claim 5, further comprising the step of identifying, via the artificial intelligence or machine learning powered module enabled with image analysis capabilities, the one or more portions of the anatomy, and potential medical defects appearing in the image related to the one or more portions of the anatomy.

7. The computerized method of claim 6, wherein the artificial intelligence or machine learning powered module is selected from the group, comprising:
machine learning models trained on various amounts of test and training data, neural networks, artificial neural networks (ANN), convolution neural networks (CNN), recurrent neural networks (RNN), deep learning models and deep-learning-based generative models, and generative adversarial networks (GANs).

8. The computerized method of claim 1, further comprising the step of using the image as augmented by the annotation to properly align the one or more portions of the anatomy of the patient.

9. The computerized method of claim 1, further comprising the steps of:
identifying, via an image analysis module, a plurality of anatomical components in the image;
placing annotations on the image associated with each of the plurality of anatomical components.

10. The computerized method of claim 9, wherein the anatomical components are selected from the group, comprising:
an ilium, an ischium, a pubis, a femoral head, an acetabulum, a femoral neck, a greater trochanter, a lesser trochanter, and a pelvic line.

11. A computerized system for annotating imaging scans for use in surgery, the system, comprising:
one or more hardware processors configured by machine readable instructions to:
receive, from a communication device, an image, wherein the image contains a visual representation of an area of a body of a patient; and
display, via a graphical user interface, the image;
provide to a user, via the graphical user interface, a set of graphical tools, wherein the set of graphical tools allow the user to annotate the image;
receive an annotation, based at least in part on input to the graphical user interface, wherein the annotation relates to one or more portions of anatomy appearing in the image;
apply the annotation to the image in a manner such that the annotation is aligned with a portion of anatomy appearing in the image.

12. The computerized system of claim 11, wherein, the image is an x-ray image.

13. The computerized system of claim 11, wherein the area of the body of a patient is one or more hips of the patient.

14. The computerized system of claim 13, wherein the one or more portions of the anatomy is selected from the group, comprising:
an ilium, an ischium, a pubis, a femoral head, an acetabulum, a femoral neck, a greater trochanter, a lesser trochanter, and a pelvic line.

15. The computerized system of claim 14, wherein the one or more hardware processors are further configured by machine readable instructions to generate, via an artificial intelligence or machine learning powered module enabled with image analysis capabilities, one or more annotations for the one or more portions of the anatomy.

16. The computerized system of claim 15, wherein the one or more hardware processors are further configured by machine readable instructions to identify, via the artificial intelligence or machine learning powered module enabled with image analysis capabilities, the one or more portions of the anatomy, and potential medical defects appearing in the image related to the one or more portions of the anatomy.

17. The computerized system of claim 16, wherein the artificial intelligence or machine learning powered module is selected from the group, comprising:
machine learning models trained on various amounts of test and training data, neural networks, artificial neural networks (ANN), convolution neural networks (CNN), recurrent neural networks (RNN), deep learning models and deep-learning-based generative models, and generative adversarial networks (GANs).

18. The computerized system of claim 11, wherein the one or more hardware processors are further configured by machine readable instructions to use the image as augmented by the annotation to properly align the one or more portions of the anatomy of the patient.

19. The computerized system of claim 11, wherein the one or more hardware processors are further configured by machine readable instructions to, comprising:
identify, via an image analysis module, a plurality of anatomical components in the image;
place annotations on the image associated with each of the plurality of anatomical components.

20. The computerized system of claim 19, wherein the anatomical components are selected from the group, comprising:
an ilium, an ischium, a pubis, a femoral head, an acetabulum, a femoral neck, a greater trochanter, a lesser trochanter, and a pelvic line.
